(19) United States
(12) Patent Application Publication
(10) Pub. No.: US 2014/0024937 A1
(43) Pub. Date: Jan. 23, 2014

(54) APPARATUS AND METHOD FOR MEASURING AN AMOUNT OF URINE IN A BLADDER

(71) Applicant: MCUBE TECHNOLOGY CO., LTD., Seoul (KR)

(72) Inventors: Jung-Hoe KIM, Seoul (KR); Seung-Tai KIM, Seoul (KR)

(73) Assignee: MCUBE TECHNOLOGY CO., LTD., Seoul (KR)

(21) Appl. No.: 14/037,930

(22) Filed: Sep. 26, 2013

Related U.S. Application Data
(63) Continuation-in-part of application No. 13/029,798, filed on Feb. 17, 2011.
Application No. 13/029,798 is a continuation-in-part of application No. 12/045,680, filed on Mar. 10, 2008, now abandoned.

(30) Foreign Application Priority Data
Apr. 25, 2006 (KR) 10-2006-0037132

(51) Int. Cl. A61B 8/08; A61B 8/00

(52) U.S. Cl. CPC A61B 8/48; A61B 8/461
USPC 600/438

(57) ABSTRACT

An apparatus for measuring an amount of urine in a bladder using ultrasonic signals comprises: a transducer; a switch for selecting one of operational modes, which include a preliminary scan mode and a scan mode; a transducer drive unit for driving the transducer; and a central control unit for operating according to the operational mode to provide an amount of urine in the bladder. The central control unit in the preliminary scan mode acquires the ultrasonic signals for a single scan plane from the transducer, generates a 2-dimensional B-Mode ultrasonic image using the acquired ultrasonic signals, displays the B-Mode ultrasonic image, and marks a vertical center-line on the B-Mode ultrasonic image. The central control unit in the scan mode acquires ultrasonic signals for a plurality of scan planes from the transducer, measures an amount of urine in the bladder using the acquired ultrasonic signals, and provides the amount of urine.

### BACKGROUND OF THE INVENTION

1. Field of the Invention

The present invention relates generally to a portable apparatus for measuring an amount of urine in a bladder using ultrasonic signals and, more particularly, to a portable and small-sized apparatus, which has a preliminary scan mode and a scan mode, thus not only quickly and accurately detecting the location of the urinary bladder but also automatically measuring the amount of urine in the urinary bladder, and a method, which can measure the amount of urine in the urinary bladder using the apparatus.

2. Description of the Prior Art

Generally, an ultrasonic system is a system that emits ultrasonic signals to an object to be examined using the piezoelectric effect of a transducer, receives the ultrasonic signals reflected from the discontinuous planes of the object, converts the received ultrasonic signals into electrical signals, and outputs the electrical signals to a predetermined display device, thus enabling examination of the internal state of the object. Such an ultrasonic system is widely used for medical diagnosis equipment, non-destructive testing equipment and underwater detection equipment.

However, most conventional ultrasonic diagnosis apparatuses are inconvenient in that they cannot be easily carried due to their large size and heavy weight. To solve the inconvenience, various portable ultrasonic diagnosis apparatuses have been proposed. Korean Utility Model Registration No. 20-137995 discloses a “Portable Ultrasonic Diagnosis Apparatus.”

Meanwhile, when examining bladder abnormalities or urinary difficulty, measuring the amount of urine is an essential procedure. Furthermore, prior to urination using a catheter, the amount of urine in the urinary bladder should be measured to account for urine that may be retained after the operation. In addition, in urination training, the amount of urine in the urinary bladder should be measured as a guideline.

Various types of ultrasonic scanning equipment may be used to measure the amount of urine in the urinary bladder, as described above. In this case, two methods are used. A first method calculates the amount of urine from respective ultrasonic images for a perpendicular plane and a horizontal plane, which are obtained using typical ultrasonic scanning equipment. However, although many algorithms has been proposed and used for the method, the first method is problematic in that it not only exhibits a considerable error rate but also exhibits different results for different users. A second method uses dedicated ultrasonic equipment for measuring the amount of urine. U.S. Pat. No. 4,926,871 discloses dedicated ultrasonic equipment. However, the dedicated ultrasonic equipment based on the second method has a disadvantage in that it also calculates the amount of urine chiefly using two ultrasonic images, which are related to the perpendicular and horizontal planes of the urinary bladder, respectively, and in that a user must find the area indicating the greatest size and select it in order to calculate the amount of urine. Further, the conventional 3-dimensional ultrasonic scanning equipments typically require long time to acquire the object and display it.

The area of the bladder acquired from a scan plane depends on the position on which the transducer is placed. FIG. 7A is a diagram illustrating the area of bladder acquired from a scan plane when the transducer of the apparatus is placed over the center of the bladder. FIG. 7B is a diagram illustrating the area of bladder acquired from the scan plane when the transducer of the apparatus is placed on the upper position which is deviated from the center of the bladder. Referring to FIGS. 7A and 7B, the cross-section areas of the bladder according to B-B′ are different each other. The volume of bladder and the amount of urine in the bladder calculated using the areas of the bladder can be varied according to the position which the transducer is placed on.

Accordingly, the apparatus and method disclosed herein are designed to overcome the above disadvantages of conventional equipments.

### SUMMARY OF THE INVENTION

Accordingly, the present invention is an apparatus for measuring an amount of urine in a bladder using ultrasonic signals, comprising: a transducer for transmitting ultrasonic signals and receiving ultrasonic signals reflected to and from the bladder and its surrounding tissues; a switch for selecting one of operational modes, which include a preliminary scan mode and a scan mode; a display unit for outputting images; a transducer drive unit for driving the transducer; and a central control unit for operating according to the operational mode selected by the switch to provide an amount of urine in the bladder, wherein the central control unit in the preliminary scan mode controls the transducer drive unit to acquire the ultrasonic signals for a single scan plane from the transducer, generates a 2-dimensional B-Mode ultrasonic image using the acquired ultrasonic signals, and displays the B-Mode ultrasonic image to the display unit, and wherein the central control unit in the scan mode acquires ultrasonic signals for a plurality of scan planes from the transducer, measures an amount of urine in the bladder using the acquired ultrasonic signals, and displays the amount of urine to the display unit.

Further, the present invention is a method for measuring and providing an amount of urine in the bladder using ultrasonic signals, comprising the steps of: (a) determining an operational mode; (b) if the operational mode is a preliminary scan mode, acquiring the ultrasonic signals for a single scan plane from a transducer, generating a 2-dimensional B-Mode ultrasonic image using the acquired ultrasonic signals, displaying the B-Mode ultrasonic image to a display unit, and marking a vertical center-line on the B-mode ultrasonic image; and, (c) if the operational mode is a scan mode, acquiring ultrasonic signals for a plurality of scan planes from the transducer, measuring the amount of urine in the bladder using the acquired ultrasonic signals, and displays the amount of urine to the display unit.

### BRIEF DESCRIPTION OF THE DRAWINGS

FIG. 1 is a block diagram schematically showing the internal construction of the apparatus according to the preferred embodiment of the present invention.

FIG. 2 is a perspective view showing a transducer and a transducer drive unit of the apparatus according to the preferred embodiment of the present invention.

FIG. 3 is a flowchart sequentially illustrating processes of measuring the amount of the urine volume in the bladder by the central control unit 100 according to the present invention.

FIG. 4 is a conceptual diagram illustrating of acquiring the ultrasonic signals for a single scan plane using the apparatus of the present invention.

FIG. 5 is an example of the 2-dimensional B-mode ultrasonic image and the vertical center-line displayed on the display unit in the preliminary scan mode according to the present invention.

FIG. 6 is a flowchart sequentially illustrating a process of measuring an amount of urine in the bladder in scan mode according to the present invention.

FIG. 7A is a diagram illustrating the area of bladder acquired from a scan plane when the transducer of the apparatus is placed over the center of the bladder.

FIG. 7B is a diagram illustrating the area of bladder acquired from a scan planes when the transducer of the apparatus is placed on the upper position which is deviated from the center of the bladder.

FIGS. 8, 9A, 9B, 9C and 9D are diagrams illustrating a bladder acquired from each of the ultrasonic scan planes according to the present invention in order to explain the procedures of measuring the volume of urine.

### DETAILED DESCRIPTION OF THE INVENTION

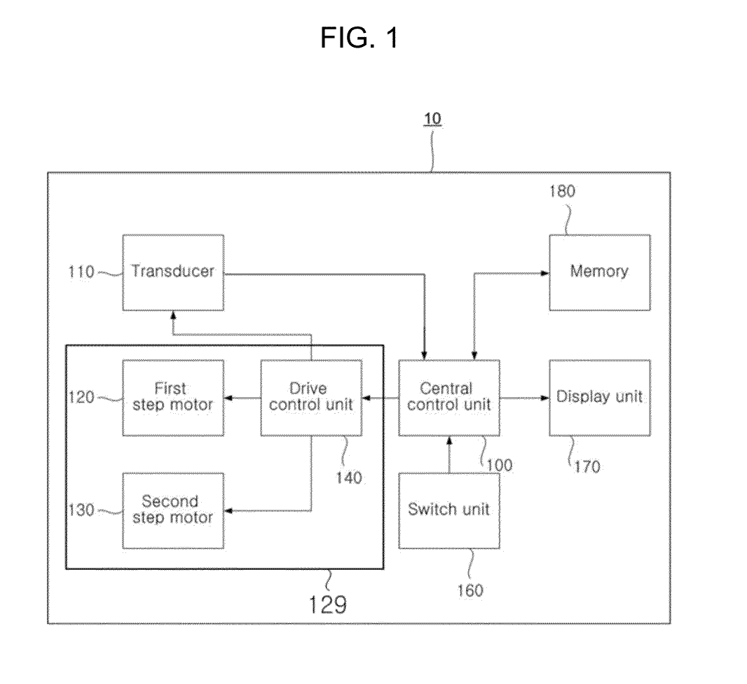

The construction and operation of an apparatus for measuring an amount of urine in a urinary bladder according to a preferred embodiment of the present invention are described in detail with reference to the accompanying drawings below. FIG. 1 is a block diagram schematically showing the internal construction of the apparatus according to the preferred embodiment of the present invention.

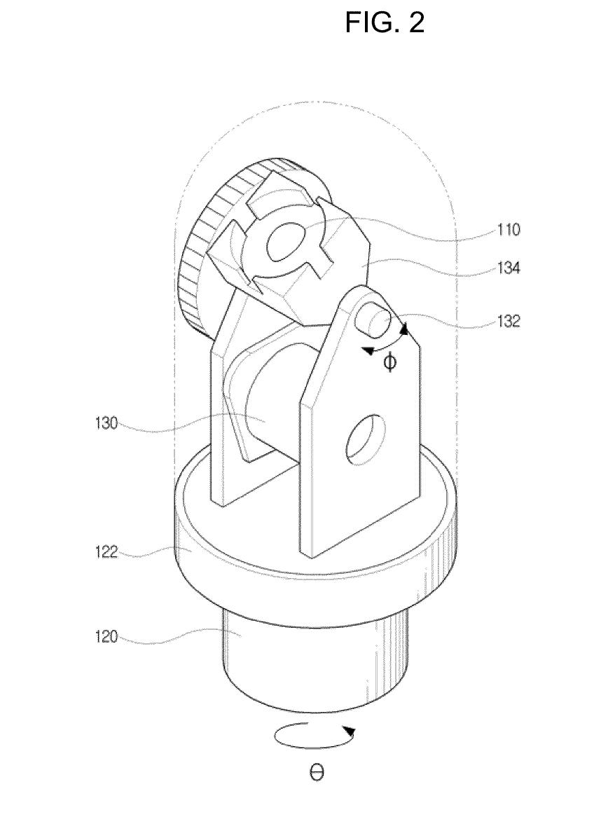

Referring to FIG. 1, the apparatus 10 according to the preferred embodiment of the present invention includes a central control unit 100 for controlling the overall operation of the apparatus, a transducer 110, a transducer drive unit 129, a switch 160, memory 180, and a display unit 170. The transducer drive unit 129 includes a first stepping motor 120, a second stepping motor 130, and a drive control unit 140. The respective components of the above-described apparatus 10 are described in detail below. FIG. 2 is a perspective view showing the transducer 110 and the transducer drive unit 129 of the apparatus according to the preferred embodiment of the present invention.

The transducer 110 is a device that transmits ultrasonic signals and receives ultrasonic signals reflected from the internal organs of a human body, and transmits the received ultrasonic signals to the central control unit 100.

The switch 160 performs input to select operational modes, such as a preliminary scan mode and a scan mode. The switch 160 according to a preferred embodiment of the present invention enables an operational mode, depending on input time or input form, to be determined using a single switch. In addition, another embodiment of the switch 160 of the present invention may be configured to be provided with a plurality of buttons, and allow different buttons to be assigned to respective operational modes.

As shown in FIG. 2, a rotational support 122 is fixed to the first stepping motor 120. A second stepping motor 130 is mounted on the rotational support 122. The second stepping motor 130 is connected with a rotational axis 132, and a transducer support 134 is fixed to the rotational axis 132. A transducer 110 is installed in the transducer support 134.

The central control unit 100 transmits drive control signals to the drive control unit 140 in response to a request signal received from the switch unit 160, and the drive control unit 140 controls the motion of the first and second stepping motors 120 and 130 in response to the drive control signals, so that the ultrasonic signals for scan planes can be obtained through the rotation of the transducer 110.

The transducer 110 permits freedom of movement along two orthogonal axes. A pair of stepping motor, those are the first and second stepping motors 120 and 130, move the transducer 110 through a predetermined path under the drive control unit 140 control.

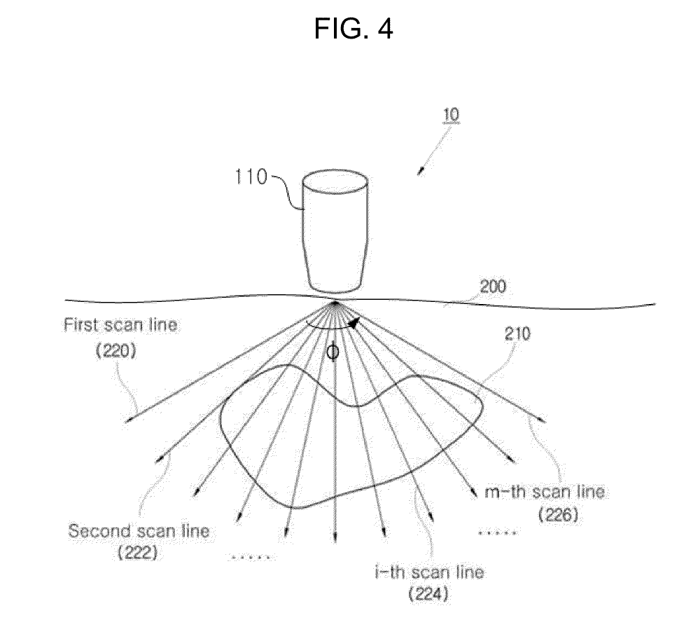

To acquire ultrasonic signals for a single scan plane, the first and second stepping motors move the transducer through three-dimensional space. FIG. 4 is a conceptual diagram illustrating of acquiring the ultrasonic signals for a single scan plane using the apparatus of the present invention. Referring to FIGS. 2 and 4, the second stepping motor 130 moves the transducer 110 in the phi(Φ) dimension of a single scan plane. The angle between two boundary edges of the scan plane, that is a total angle phi(Φ), can vary, but typically will be approximately 120°. The rotational axis 132 and the transducer support 134, which are connected to the second stepping motor 130 using a connection unit, are rotated by the second stepping motor 130. The connection unit can be used a gear, a belt or etc.

Consequently, the transducer 110 installed in the transducer support 134 rotates in the phi(Φ) dimension of a single scan plane.

To acquire ultrasonic signals for a plurality of scan planes, the first stepping motor 120 rotates the transducer assembly about a central vertical axis through a total angle theta(θ) in series of small angular movements. Typically, the total angle theta will be 360°. The first stepping motor rotates successively a predetermined angle in the theta direction, at each theta position a plurality of ultrasonic signals for a scan plane are acquired according to the above-described process. The total number of scan planes over the entire theta dimension of 360° will completely sample the ultrasonic scan cone by ultrasonic signals. The ultrasonic signals are processed to determine the locations of the bladder walls in each of the scan planes.

The central control unit 100 determines an operational mode based on a signal input through the switch 160. Thereafter, when the preliminary scan mode is determined, an operation is performed in the preliminary scan mode. In contrast, when the scan mode is determined, an operation is performed in the scan mode.

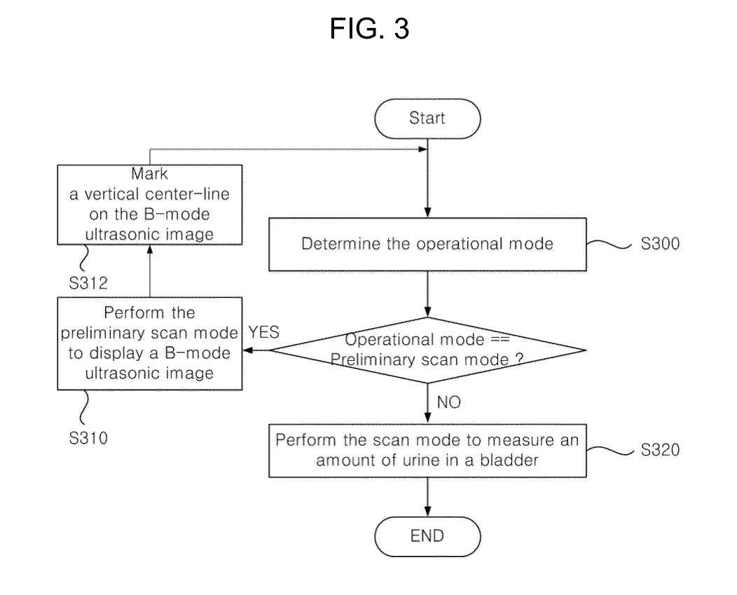

FIG. 3 is a flowchart sequentially illustrating processes of obtaining the amount of the urine volume in the bladder by the central control unit 100 according to the present invention. Referring to FIG. 3, the central control unit 100 of the present invention determines an operational mode, at step 300. If the operational mode is a preliminary scan mode, the central control unit 100 obtains the ultrasonic signals for a single scan plane from a transducer, generating a 2-dimensional B-Mode ultrasonic image using the obtained ultrasonic signals, and displaying the B-Mode ultrasonic image to a display unit, at step 310. Then, the central control unit 100 marks a vertical center-line on the B-Mode ultrasonic image to make an operator determine easily the position of bladder on the B-Mode ultrasonic image, at step 312.

If the operational mode is a scan mode, the central control unit 100 obtains ultrasonic signals for a plurality of scan planes from the transducer, measuring an amount of urine in the bladder using the obtained ultrasonic signals, generates 2-dimensional B-mode images using the obtained ultrasonic signals, displays the generated 2-dimensional B-mode images to the display unit, and displays the amount of urine to the display unit, at step 320. The central control unit performs repeatedly the steps 300, 310, 312 and 320.

The operation in the preliminary scan mode of the ultrasonic measurement apparatus according to the present invention are described below.

The central control unit 100 in the preliminary scan mode controls the transducer drive unit 129 to control the movement of the transducer and receive the ultrasonic signals for a single scan plane from the transducer, compensates the received ultrasonic signals using the predetermined time-controlled gains, generates a 2-dimensional B-Mode ultrasonic image using the compensated ultrasonic signals, displays the 2-dimensional B-Mode ultrasonic image to the display unit, and marks a vertical center-line on the 2-dimensional B-Mode ultrasonic image.

To acquire the ultrasonic signals for a single scan plane, the central control unit transmits a drive control signal to the drive control unit of the transducer drive unit 129. The drive control unit rotates the second stepping motor in a yz direction (that is, a second direction) in response to the drive control signal received from the central control unit. As the second stepping motor rotates, the transducer fixed on the second stepping motor also rotates.

Referring to FIG. 4, the transducer acquires the pieces of ultrasonic signals of n scan lines 220, 222, . . . , 226 for a single scan plane in the yz direction while rotating in the yz direction. Meanwhile, the central control unit receives the pieces of ultrasonic signals of n scan lines for a single scan plane from the transducer, generates a 2-dimensional B-mode ultrasonic image using the ultrasonic signals for a single scan plane, and outputs the 2-dimensional B-mode ultrasonic image to the display unit. In this case, in the state in which the transducer is disposed on the abdomen of a patient and is oriented toward his or her urinary bladder in the preliminary scan mode, the transducer of the apparatus according to the present invention rotates in left and right directions or in up and down directions relative to the patient, that is, a lateral direction with respect to the patient, and thus a two-dimensional B-mode ultrasonic image obtained as a result of the rotation is output to the display unit.

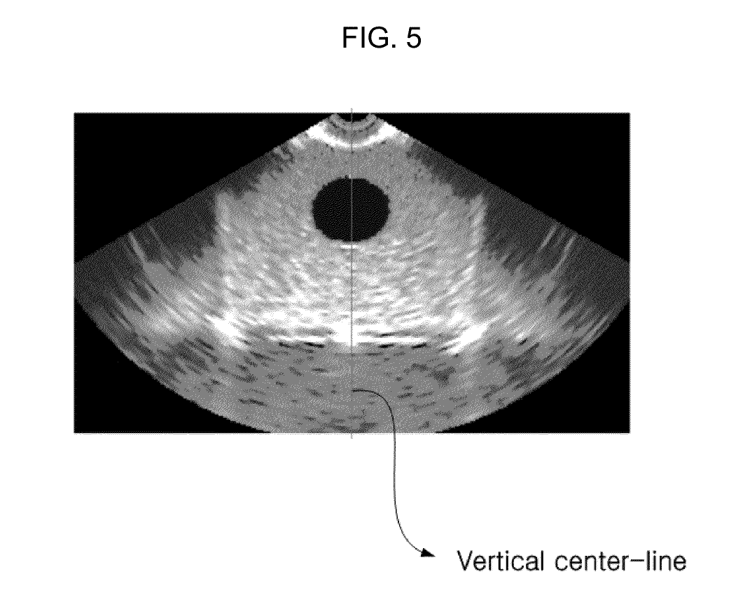

FIG. 5 is an example of the 2-dimensional B-mode ultrasonic image displayed on the display unit in the preliminary scan mode according to the present invention.

A operator, who operates the apparatus according to the present invention, causes the apparatus to operate in the preliminary scan mode and then views the B-mode ultrasonic image and the vertical center-line on the display unit, so that he or she can be quickly and accurately made aware of the location of the urinary bladder which is to be examined.

Furthermore, in the preliminary scan mode, the above-described process is periodically repeated until the scan mode is selected by the switch and a two-dimensional B-mode ultrasonic image for a corresponding plane is output to the display unit. In this case, the repetition period of acquiring and displaying the two-dimensional B-mode ultrasonic image in the preliminary scan mode is less than about 0.5 second. Accordingly, the apparatus of the present invention can provide quickly the two-dimensional B-mode ultrasonic image for aiming the transducer.

Meanwhile, in another embodiment of the apparatus according to the present invention for the preliminary scan mode, when the preliminary scan mode is selected, two-dimensional B-mode ultrasonic images for more than two scan planes are acquired and are displayed on the display unit. In this case, it is preferred that the more than two scan planes of two-dimensional B-mode ultrasonic images are formed to have different orientation angles.

The operation in the scan mode of the ultrasonic measurement apparatus according to the present invention are described below. The central control unit 100 in the scan mode controls the transducer drive unit 129 to obtain ultrasonic signals for a plurality of scan planes from the transducer, generates 2-dimensional B-mode images using the obtained ultrasonic signals, displays the generated 2-dimensional B-mode images to the display unit, measures an amount of urine in the bladder ultrasonic signals for a plurality of scan planes, and displays the amount of urine to the display unit.

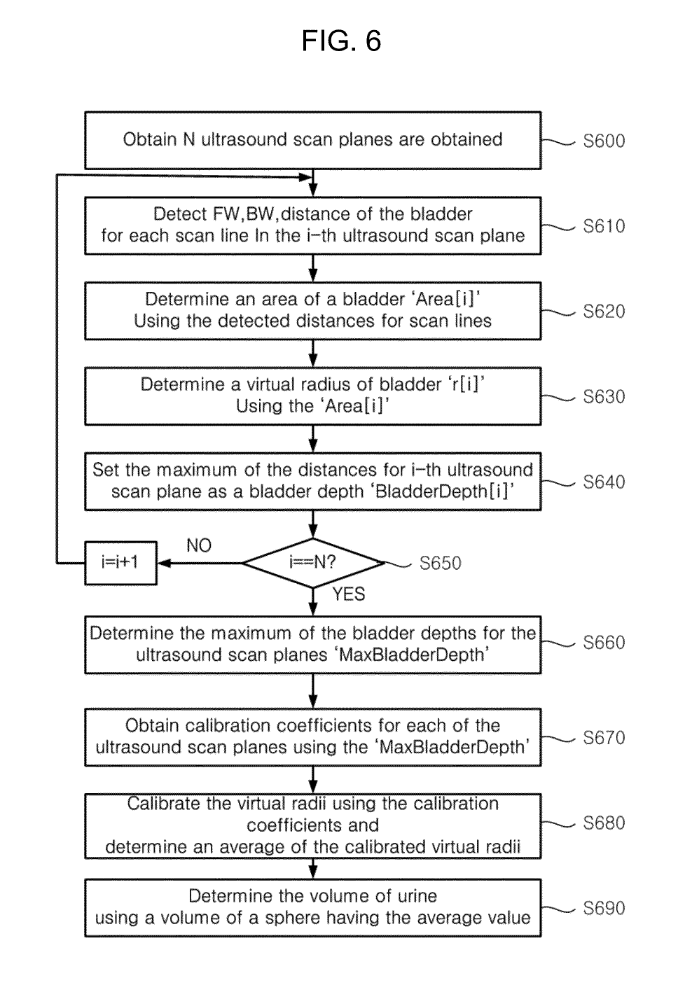

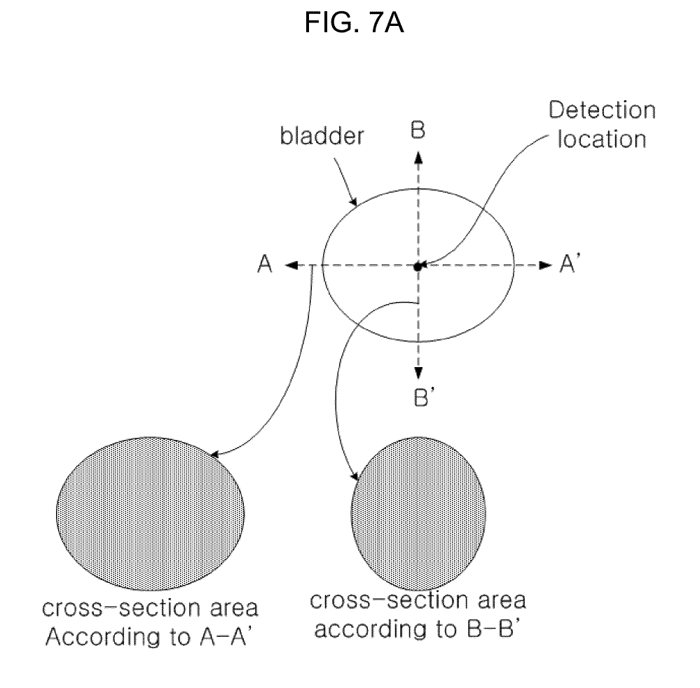

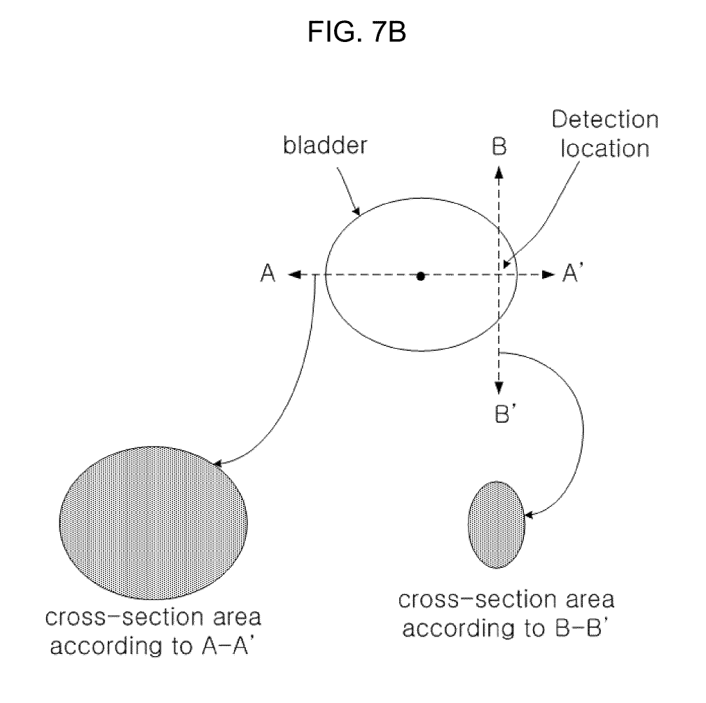

The method for measuring the amount of urine in the bladder according to the present invention is described below. FIG. 6 is a flowchart sequentially illustrating a process of measuring an amount of urine in the bladder in scan mode according to the present invention.

Referring to FIG. 6, the central control unit receives ultrasonic signals for a plurality of scan planes from the transducer, which each of the scan planes is separated by a selected angle and consists of a plurality of scan lines to produce a scan cone for scanning the bladder, at step 600.

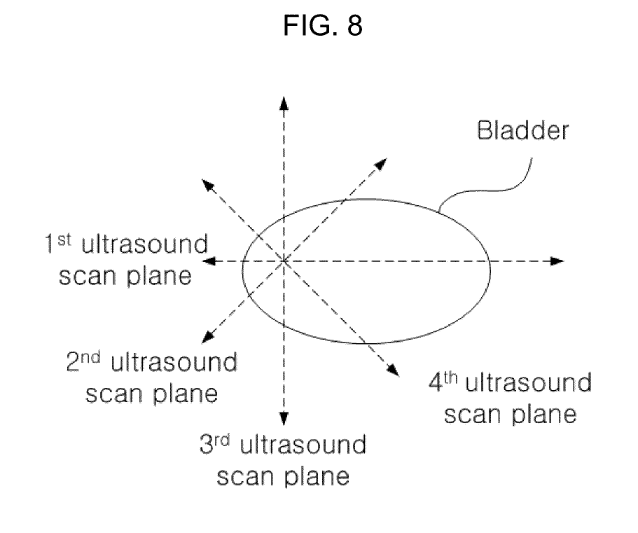

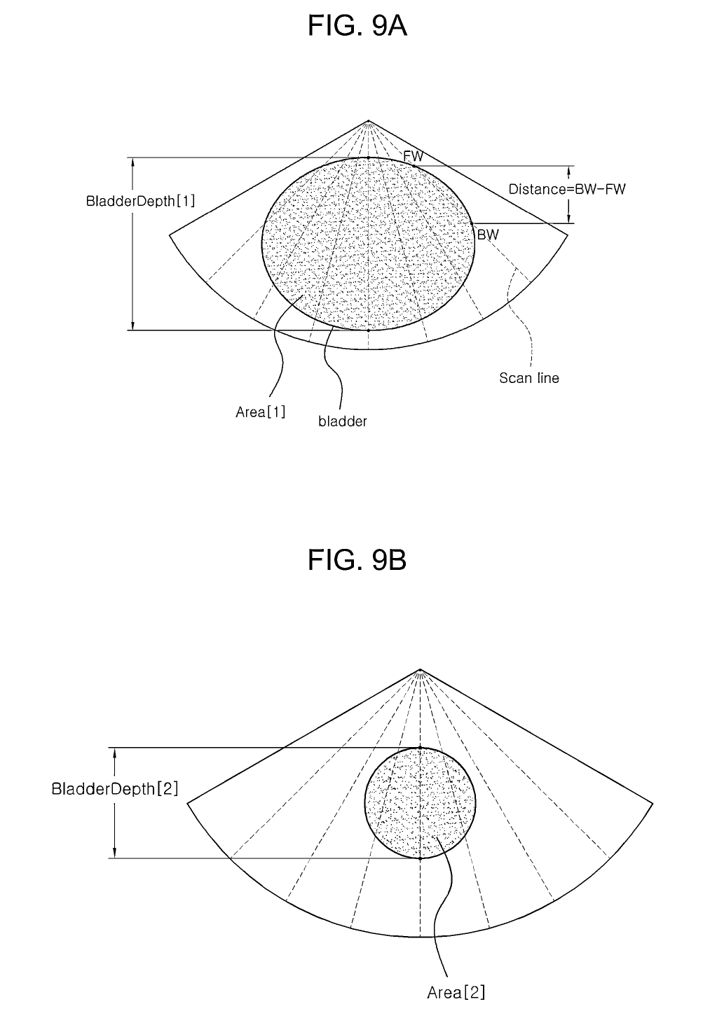

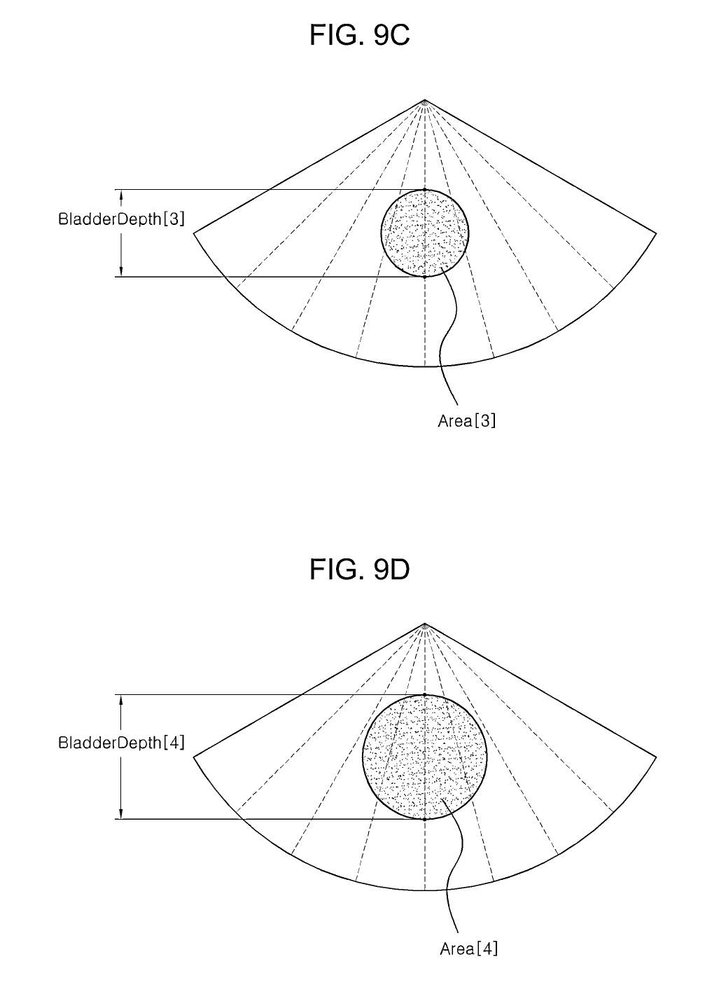

FIG. 8 represents the 4 scan directions of the transducer to obtain 4 ultrasonic scan planes, respectively. FIGS. 9A, 9B, 9C and 9D represent 1st, 2nd, 3rd and 4th scan planes according to FIG. 8, respectively. Referring to FIGS. 8, 9A, 9B, 9C and 9D, the ultrasonic signals of scan line contains information about the border of the bladder, specified as FW (Front wall) and BW (Back wall) of the bladder, and the distance between FW and BW of the bladder. The remaining portion of the ultrasonic signals in the scan lines is the returning signal from the surrounding tissues.

Referring to FIG. 9A, the distances between FW and BW of the bladder, ‘Distance’, are detected from ultrasonic signals of each of the scan lines constituting the i-th ultrasonic scan plane at step 610.

At next step, the area of the bladder in the i-th ultrasonic scan plane, Area[i], is obtained using the distance values for the scan lines constituting the i-th ultrasonic scan plane at step S620. In this case, the method of obtaining an area of the bladder may be implemented in various ways. As an example, a method of obtaining the area of a bladder may be implemented by totaling areas for sectors, each of the sectors is formed by rotating a single scan line using the rotational angle of the second stepping motor 130. As another example, a method of obtaining the area of the bladder may be implemented by totaling the areas for trapezoids, each of the trapezoids is formed by the two front walls and two back walls of the neighbored scan lines.

The virtual radius of the bladder in i-th scan plane ‘r[i]’ is generated using the obtained area of the bladder ‘Area[i]’. Given the assumption that the bladder is a circle, the virtual radius ‘r[i]’ is determined by a radius of a circle of which the area is same as the area of the bladder ‘Area[i]’ at step S630.

Typically, in the case of obtaining a three-dimensional volume using a plurality of two-dimensional areas, the volume smaller than an actual volume is calculated and, thus, an error occurs if scanning is performed in a state in which the center of a first rotational axis deviates from the center of the bladder. Accordingly, numerical calibration procedure is performed to reduce such error and accurately measure the volume of urine in the bladder.

A bladder depth of the bladder in i-th scan plane ‘BladderDepth[i]’ is determined by the maximum value of the distances for i-th scan plane at step S640.

The steps S610, S620, S630 and S640 are repeatedly implemented to all of the scan planes. Thereafter, the maximum value of the bladder depths for the scan planes ‘MaxBladderDepth’ is obtained at step S660. Thereafter, at step S670, the calibration coefficient for each of the scan planes is obtained using the following equation 1:

$$ComFactor [ i ] = \frac{MaxBladderDepth}{BladderDepth [ i ]} \qquad ( 1 )$$

Where ComFactor[i] is the calibration coefficient for i-th scan plane, BladderDepth[i] is the bladder depth for i-th scan plane.

Thereafter, at step S680, calibrated radius of the bladder for each of the ultrasonic scan planes is calculated using the following Equation 2:

ComR[i]=ComFactor[i]×r[i] (2)

Where, ‘ComR[i]’ is the calibrated radius of the bladder for i-th scan plane.

The average value of the calibrated radii of the bladders for the scan planes ‘AverageR’ is obtained at step S680. Thereafter, given the assumption that the complete bladder is a sphere, the total volume of urine ‘V’ in the bladder by applying the average radius ‘AverageR’ to the following Equation 3 is obtained at step S690.

V=4/3πAverageR³ (3)

From the above-described process, the method for measuring the volume of urine in a bladder according to the present invention can accurately detect the volume of urine in the bladder although the transducer is placed on the position which is moved from center of the bladder.

Furthermore, the apparatus of the present invention collects the ultrasonic signals while automatically rotating the two stepping motors, so that it can collect all pieces of ultrasonic information within a region defined in a cone shape having a vertex at the location at which the apparatus according to the present invention is disposed. As a result, the apparatus of the present invention can very accurately measure the volume of urine using ultrasonic information about a plurality of ultrasonic scan planes that are spaced apart from each other and exist in an angle of 360°.

The number of scan planes to be scanned and the number of scan lines for a single plane may be determined according to the region and size of the object to be examined. In the case of measuring the urinary bladder, the number of scan lines and the number of scan planes may be determined such that the entire region of the urinary bladder can be included. For example, in the case of scanning the urinary bladder, the entire region of the urinary bladder can be sufficiently included using about 67 scan lines if the angle between scan lines for forming a single B-mode ultrasonic image is 1.8°.

In particular, the apparatus and the method of the present invention calibrate the radii of the bladder using the calibration coefficients, which are obtained by calculating the degree to which a first detection location is moved from the center of the urinary bladder, so that it can perform accurate measurement even when the detection location is moved from the center of the urinary bladder.

Although the present invention has been described in detail in conjunction with the preferred embodiment, the present invention is described only for illustrative purposes and is not limited thereto. Those skilled in the art will appreciate that various modifications and applications, which are not described above, are possible within a range that does not change the substantial characteristics of the present invention. For example, in the present embodiment, the method of obtaining an area of a bladder for a corresponding plane using the rotational angles of the first stepping motor and the second stepping motor and ultrasonic information about the respective scan lines may be modified and implemented in various ways to improve scanning performance. Furthermore, it should be appreciated that the differences regarding the modifications and the applications are included in the scope of the present invention, which is defined by the accompanying claims.

What is claimed:

1. An apparatus for measuring an amount of urine in a bladder using ultrasonic signals, comprising:
a transducer for transmitting ultrasonic signals and receiving ultrasonic signals reflected to and from the bladder and its surrounding tissues;
a switch for selecting one of operational modes, which include a preliminary scan mode and a scan mode;
a display unit for outputting images;
a transducer drive unit for driving the transducer; and
a central control unit for operating according to the operational mode selected by the switch to provide an amount of urine in the bladder,
wherein the central control unit in the preliminary scan mode controls the transducer drive unit to obtain the ultrasonic signals for a single scan plane from the transducer, generates a 2-dimensional B-mode ultrasonic image using the obtained ultrasonic signals, displays the B-mode ultrasonic image to the display unit, and marks a vertical center-line on the B-Mode ultrasonic image, and
wherein the central control unit in the scan mode controls the transducer drive unit to obtain ultrasonic signals for a plurality of scan planes from the transducer, measures the amount of urine in the bladder using the obtained ultrasonic signals, and displays the amount of urine to the display unit.

2. The apparatus for measuring an amount of urine according to claim 1, wherein the central control unit in the preliminary scan mode compensates the received ultrasonic signals from the transducer using predetermined time-controlled gains, generates the 2-dimensional B-mode ultrasonic image using the compensated ultrasonic signals, and marks the vertical center-line on the 2-dimensional B-Mode ultrasonic image.

3. The apparatus for measuring an amount of urine according to claim 1, wherein the central control unit in the scan mode generates 2-dimensional B-mode ultrasonic images using the obtained ultrasonic signals, displays the B-mode ultrasonic images to the display unit, and marks a vertical center-line on the B-Mode ultrasonic images.

4. The apparatus for measuring an amount of urine according to claim 1, wherein the central control unit in the scan mode
receives ultrasonic signals for a plurality of scan planes from the transducer, which each of the scan planes is separated by a selected angle and consists of a plurality of scan lines to produce a scan cone for scanning the bladder,
detects distances between front wall FW and back wall BW of the bladder for each scan line in the scan planes,
calculates areas of the bladder for each scan plane using the detected distances of the scan lines,
generates virtual radii of the bladder for each scan plane using the calculated areas,
determines calibration coefficients for each scan plane using the detected distances of the scan lines,
calibrates virtual radii of the bladder for each scan plane using the calibration coefficients for the corresponding scan planes,
determines the volume of the bladder using the calibrated virtual radii for the scan planes.

5. The apparatus for measuring an amount of urine according to claim 4, wherein the calibration coefficients for each scan plane are calculated using the following equation:
$$ComFactor [ i ] = \frac{Max bladderDepth}{BladderDepth [ i ]}$$
where ComFactor[i] is a calibration coefficient for an i-th scan plane, BladderDepth[i] is the bladder depth for the i-th scan plane and is determined by the maximum of distances between FW and BW of bladder in scan lines for the i-th scan plane, MaxBladderDepth is determined by the maximum of the bladder depths for the scan planes.

6. The apparatus for measuring an amount of urine according to claim 4, wherein the volume of the bladder is determined by calculating the volume of a sphere of which the radius is the average of the calibrated virtual radii for the scan planes.

7. The apparatus for measuring an amount of urine according to claim 4, wherein the virtual radii of the bladder for each ultrasonic scan plane are determined by the radius of a circle of which the area is the same as the detected area of the bladder for the corresponding scan plane.

8. The apparatus for measuring an amount of urine according to claim 4, wherein the virtual radii are calibrated using the following equation:
ComR[d]=ComFactor[i]×r[i]
Where, ‘ComR[i]’ is a calibrated virtual radius of the bladder for i-th scan plane, ‘ComFactor[i]’ is a calibration coefficient for an i-th scan plane, and ‘r[i]’ is a virtual radius of the bladder for i-th scan plane.

9. A method for measuring an amount of urine in the bladder using ultrasonic signals, comprising the steps of:
(a) determining an operational mode;
(b) if the operational mode is a preliminary scan mode, receiving the ultrasonic signals for a single scan plane from a transducer, generating a 2-dimensional B-mode ultrasonic image using the received ultrasonic signals, displaying the B-mode ultrasonic image to a display unit, and marking a vertical center-line on the B-Mode ultrasonic image; and,
(c) if the operational mode is a scan mode, receiving ultrasonic signals for a plurality of scan planes from the transducer, calculating the amount of urine in the bladder using the received ultrasonic signals, and displaying the amount of urine to the display unit.

10. The method for measuring an amount of urine according to claim 9, wherein the step (b) compensates the received ultrasonic signals from the transducer using predetermined time-controlled gains and generates the 2-dimensional B-mode ultrasonic image using the compensated ultrasonic signals.

11. The method for measuring an amount of urine according to claim 9, wherein the step (c) generates 2-dimensional B-mode ultrasonic images using the received ultrasonic signals, displays the B-mode ultrasonic images to a display unit, and marks a vertical center-line on the B-Mode ultrasonic images.

12. The method for measuring an amount of urine according to claim 9, wherein the step (c) comprises the steps of:
(c1) receiving ultrasonic signals for a plurality of scan planes from the transducer, which each of the scan planes is separated by a selected angle and consists of a plurality of scan lines to produce a scan cone for scanning the bladder,
(c2) detecting distances between front wall FW and back wall BW of the bladder for each scan line in the scan planes,
(c3) calculating areas of the bladder for each scan plane using the detected distances of the scan lines,
(c4) generating virtual radii of the bladder for each scan plane using the calculated areas,
(c5) determining calibration coefficients for each scan plane using the detected distances of the scan lines,
(c6) calibrating virtual radii of the bladder for each scan plane using the calibration coefficients for the corresponding scan planes,
(c7) determining the volume of the bladder using the calibrated virtual radii for the scan planes.

13. The method for measuring an amount of urine according to claim 12, wherein the calibration coefficients for each scan plane are calculated using the following equation:
$$ComFactor [ i ] = \frac{Max bladderDepth}{BladderDepth [ i ]}$$
where ComFactor[i] is a calibration coefficient for an i-th scan plane, BladderDepth[i] is the bladder depth for the i-th scan plane and is determined by the maximum of distances between FW and BW of bladder in scan lines for the i-th scan plane, MaxBladderDepth is determined by the maximum of the bladder depths for the scan planes.

14. The method for measuring an amount of urine according to claim 12, wherein the volume of the bladder is determined by calculating the volume of a sphere of which the radius is the average of the calibrated virtual radii for the scan planes.

15. The method for measuring an amount of urine according to claim 12, wherein the virtual radii of the bladder for each ultrasonic scan plane are determined by the radius of a circle of which the area is the same as the detected area of the bladder for the corresponding scan plane.

16. The method for measuring an amount of urine according to claim 12, wherein the virtual radius is calibrated using the following equation:
ComR[d]=ComFactor[i]×r[i]
